(19) United States
(12) Patent Application Publication
(10) Pub. No.: US 2018/0008944 A1
(43) Pub. Date: Jan. 11, 2018

(54) Mixing Device

(71) Applicant: ULVAC, INC., Chigasaki-shi, Kanagawa (JP)

(72) Inventor: Tomomitsu OZEKI, Kanagawa (JP)

(21) Appl. No.: 15/544,464

(22) PCT Filed: Oct. 7, 2015
(86) PCT No.: PCT/JP2015/005094

§ 371 Date: Jul. 18, 2017

(30) Foreign Application Priority Data
Jan. 19, 2015 (JP) 2015-008100

(51) Int. Cl. B01F 13/10; G01N 33/536; B01F 7/16; B01F 7/00; B01F 15/00; G01N 35/02; G01N 1/38

(52) U.S. Cl. CPC B01F 13/1022; G01N 35/026; G01N 33/536; G01N 1/38; B01F 7/00841; B01F 15/00389; B01F 7/1665; B01F 2215/0037

(57) ABSTRACT

[Object] To provide a mixing device capable of accurately mixing a solution in a multi-well plate.

[Solving Means] A mixing device 1 is configured to be attachable to a multi-well plate 30 and includes a casing 100, a plurality of stirrers 11, a plurality of motors 12 as a drive portion, and a mounting portion 16. The casing 100 includes a main surface portion 101 facing an upper surface 301 of the multi-well plate 30. The stirrers 11 protrude from the main surface portion 101 toward wells 31 of the multi-well plate 30. The motors 12 are disposed to the casing 100 and rotate the stirrers 11 about axes thereof. The mounting portion 16 is provided to the casing 100 and is mounted to the multi-well plate 30 to position the casing 100 on the multi-well plate 30.

### TECHNICAL FIELD

The present invention relates to a mixing device for mixing a solution in a well plate.

### BACKGROUND ART

Multi-well plates are also referred to as microplates, micro-well plates, microtiter plates, or the like and are widely used as an experimental or testing instrument in the field of study in medical science, pharmaceutics, biochemistry, chemistry, and the like. The multi-well plate generally has 6, 24, 96, 384, or 1536 wells and can contain approximately 1 microliter to several milliliters of a reaction solution in each of the wells. In order to detect the solution after the reaction, a plate reader is used. Additionally, an automatic solution addition and suction apparatus for adding a solution and washing wells, a conveyance system for conveying a plate in itself, and the like are commercially available as general-purpose products from various manufacturers. Because of such a background, the size of the multi-well plate is standardized at the present day. An outer circumference, well positions, a plate thickness, and the like are prescribed by the American National Standards Institute (ANSI) and the Society for Laboratory Automation and Screening (SLAS) (Non-patent Document 1).

As one of general use applications of the multi-well plate, there is an assay method in which molecules are fixed to a solid-phase support medium on the bottom surface of the multi-well plate directly or via a dedicated reagent, like ELISA (Enzyme-Linked ImmunoSorbent Assay). In ELISA, in general, a sample solution containing a target substance, an antibody, a labeled secondary antibody, and a substrate solution are sequentially added to an antibody fixed to a solid-phase support medium, and light emission or absorption is then measured, to thus determinate quantity of the target substance. In general, the substance solutions are left to stand after the addition. However, the rate of adsorption of each molecule depends on the rate of diffusion in a solution, and it is thought that the adsorption of molecules is slow in the standing method. Thus, in general, it is necessary to wait several hours to approximately half a day after the antibody or the sample solution is added.

A cell-based assay for evaluation of function on a cell-by-cell basis has recently attracted attention. Also in this case, the multi-well plate is heavily used. For a measurement format used in the cell-based assay, a 96-well plate is predominant, and additionally a 16-well plate and a 384-well plate are also used (Non-patent Document 2). Meanwhile, as described in Patent Document 2, it is important to control mixing in cell culturing and measurement. At that time, it is necessary to mix a solution without damaging cells adhering to the bottom surface or floating, and to perform highly accurate mixing. In vortex mixing, accuracy of mixing and efficiency thereof are controversial. There is mixing using a magnetic stirrer, but such mixing is not favorable because of physical contact with cells adhering to the wells.

As a mixing method for a multi-well plate, there are known a method of circularly moving the entire plate in a horizontal direction, a method of using a magnetic stirrer, and a method of using ultrasonic vibration.

The method of circularly moving the entire plate is also called vortex mixing and is well known as a simplified mixing method (Patent Document 1). In the vortex mixing, it is necessary to increase a diameter of the circular movement or increase a rotational speed in order to obtain high mixing efficiency. However, there are limitations because of structural restriction of a device in itself, a problem of noise, a problem of splashes of liquid, a problem of a load on a motor to be used, and resultant mixing efficiency is never high. Further, it is suggested that the mixing efficiency differs between the outer side and the inner side of the plate, and the vortex mixing is not suitable to uniformly accurately mix the solution in all the wells of the plate. Further, since mixing conditions on a well-by-well basis cannot be set as a matter of course, the vortex mixing is unsuitable for a test that requires detailed examination of the mixing conditions.

As in Patent Document 3, mixing with a magnetic stirrer is used in some cases, but such mixing has a narrow set range of the number of mixing rotations, and rotation is not easy to perform at low speed or high speed. Further, such mixing has low reliability in movement of the stirrer, and in the case where rotation following performance is poor, this leads to reduction in mixing accuracy and mixing efficiency. Further, there is also a problem in large size of a device in itself. Further, since it is necessary to put a magnetic tip on the bottom surface, in the case where a substance, a cell, or the like is firmly fixed to the bottom surface, there is a risk that those substances are broken or damaged. Further, mixing conditions for all the wells are uniformly determined. In order to examine many mixing conditions in detail, it is favorable to set the number of mixing rotations corresponding to each of the wells. Patent Document 4 discloses a mixing method using ultrasonic waves. The mixing method requires a mechanism for transmitting vibrations in a gap with a well plate and also needs to increase output in order to perform efficient mixing but causes a problem of temperature rise. Further, there is a problem that mixing conditions for the wells are uniformly determined.

Patent Document 1: Japanese Patent Application Laid-open No. 2007-237174

Patent Document 2: Japanese Patent Application Laid-open No. 2010-178734

Patent Document 3: Japanese Patent Application Laid-open No. 2008-241640

Patent Document 4: Japanese Patent Application Laid-open No. 2007-117830

Non-patent Document 1: http://www.slas.org/default/assets/File/ANSI_SLAS_4-2004_WellPositions.pdf

Non-patent Document 2: Drug Discovery World Summer 2008, 77-88pp “Progress in the implementation of Label-free-detection, part-1: Cell-based assays”

### SUMMARY OF INVENTION

### Problem to be Solved by the Invention

In view of the circumstances as described above, it is an object of the present invention to provide a mixing device capable of accurately mixing a solution in a multi-well plate.

### Means for Solving the Problem

In order to achieve the object described above, according to an embodiment of the present invention, there is provided a mixing device that is a mixing device configured to be attachable to a multi-well plate, the mixing device including a casing, at least one stirrer, a drive portion, and a mounting portion.

The casing includes a main surface portion facing an upper surface of the multi-well plate.

The stirrer protrudes from the main surface portion toward a well of the multi-well plate.

The drive portion is disposed on the casing and rotates the stirrer about an axis thereof.

The mounting portion is provided to the casing and is mounted to the multi-well plate to position the casing on the multi-well plate.

The mixing device is disposed on the upper surface of the multi-well plate, and the stirrer is disposed in the well of the multi-well plate from the main surface portion of the casing. The drive portion rotates the stirrer about the axis thereof and mixes a solution housed in the well by the stirrer. When the casing is disposed on the upper surface of the multi-well plate, the mounting portion is mounted at a predetermined position of the multi-well plate and positions the casing on the multi-well plate.

According to the mixing device, controlling rotation (the number of rotations or rotational speed) of the stirrer enables the solution in the well to be efficiently mixed. Further, mounting of the mounting portion to the multi-well plate highly accurately positions the stirrer with respect to the well, and thus stable mixing accuracy can be achieved irrespective of the size of the well.

The mixing device typically includes a plurality of stirrers. In this case, the mounting portion is mounted to the multi-well plate to position the plurality of stirrers into predetermined wells of the multi-well plate.

According to the mixing device, mounting of the mounting portion to the multi-well plate can accurately position the plurality of stirrers with respect to the respective wells.

The plurality of stirrers may be disposed to correspond to all wells of the multi-well plate or may be disposed to correspond to some wells (for example, a plurality of wells belonging to a predetermined row of the multi-well plate). In other words, the number of stirrers is not limited to a case corresponding to the number of wells of the multi-well plate.

A mounting position of the mounting portion with respect to the multi-well plate is not particularly limited. A configuration of the mounting portion can also be appropriately set in accordance with the mounting position.

For example, the mounting portion includes a space portion that is configured to be capable of housing the multi-well plate, and an engaging surface that comes into contact with an outer circumferential surface of the multi-well plate housed in the space portion or a part of the outer circumferential surface.

Alternatively, the mounting portion includes a plurality of engaging protrusions that are configured to be engaged with predetermined wells of the multi-well plate.

Alternatively, the mounting portion may be constituted of a frame body configured to be separable from the casing. The frame body has an inner circumferential surface that is engageable with an outer circumferential portion of the casing and an outer circumferential portion of the multi-well plate.

The mixing device may further include a sheet member that is provided to the main surface portion and can elastically come into contact with the upper surface of the multi-well plate. With this configuration, adhesion between the main surface portion and the multi-well plate is increased, and even if the solution to be mixed is volatile, evaporation of the solution during mixing can be suppressed.

The drive portion may include a plurality of motors that are respectively attached to the plurality of stirrers. In this case, the mixing device may further include a controller. The controller is configured to individually control drive of the plurality of motors.

With this configuration, the individual stirrers can be independently rotated. Each of the stirrers may be driven under the same rotation condition or different rotation conditions.

The drive portion may be disposed in an internal space of the casing, and the mixing device may further include a fan that is disposed in the internal space. With this configuration, a drive source can be cooled by an airflow generated by the fan.

The mixing device may further include a fan mounting plate that partitions the internal space into a first internal space and a second internal space and has an opening that causes the first internal space and the second internal space to communicate with each other, the first internal space housing the drive portion, the second internal space housing the fan, and the fan may generate an airflow flowing between the first internal space and the second internal space via the opening. With this configuration, an airflow flowing between the first internal space housing the drive portion and the second internal space housing the fan can be generated.

The casing may include a first vent and a second vent, the first vent causing the first internal space and an outer space of the casing to communicate with each other, the second vent causing the second internal space and the outer space to communicate with each other. With this configuration, it is possible to generate an airflow flowing in the first internal space from the outer space via the first vent, flowing in the second internal space via the opening of the fan mounting plate, and outflowing to the outer space from the second vent, and to cool the drive portion.

The mounting portion may be an attachment configured to be separable from the casing. Further, the mounting portion may be mounted to the multi-well plate via a positioning member mounted to the multi-well plate.

The mixing device may further include a sheet member that elastically comes into contact with the main surface portion, the drive portion may include a chassis, a rotary shaft, and a bearing, the chassis housing a rotor and a stator, the rotary shaft being connected to the rotor, the bearing being fixed to the chassis and rotatably supporting the rotary shaft, the stirrer may be connected to the rotary shaft, and the mixing device may further include a sealing that seals a gap between the sheet member and the stirrer. The sealing can prevent vapor of the liquid to be mixed from reaching the bearing and prevent grease from outflowing or degrading.

The mixing device may further includes a sheet member that elastically comes into contact with the main surface portion, the drive portion may include a chassis, a rotary shaft, and a bearing, the chassis housing a rotor and a stator, the rotary shaft being connected to the rotor, the bearing being fixed to the chassis and rotatably supporting the rotary shaft, the stirrer may be connected to the rotary shaft, and the casing may seal a gap between the sheet member and the stirrer. Using the casing can also prevent vapor of the liquid to be mixed from reaching the bearing and prevent grease from outflowing or degrading.

The mixing device may further include a controller that controls the drive portion, the controller controlling the drive portion to generate a first torque for a certain period of time when the drive portion starts to rotate and controlling the drive portion to alternately generate a second torque and the first torque after the certain period of time elapses, the second torque being smaller than the first torque. When the drive portion starts to rotate, the drive portion can be caused to generate the first torque to reliably rotate the stirrers, and during the rotation, caused to generate a smaller second torque to prevent heat generation due to the drive portion. Further, the first torque is periodically generated, and thus if the rotation of the stirrer is stopped, the rotation can be restarted.

### Effects of the Invention

As described above, according to the present invention, it is possible to accurately mix a solution in a multi-well plate.

### BRIEF DESCRIPTION OF DRAWINGS

FIG. 1 is a perspective view of a mixing device according to a first embodiment of the present invention.

FIG. 2 is a cross-sectional view of a main part of the mixing device.

FIG. 3 is a cross-sectional view of the main part in a state where the mixing device is attached to a multi-well plate.

FIG. 4 is a perspective view of a configuration of a mixing device according to a second embodiment of the present invention.

FIG. 5 is a cross-sectional view of a main part showing a configuration of a mixing device according to a third embodiment of the present invention.

FIG. 6 is a perspective view of a configuration of a mixing device according to a fourth embodiment of the present invention.

FIG. 7 is a cross-sectional view of a main part showing a configuration of a mixing device according to a fourth embodiment of the present invention.

FIG. 8 is a schematic cross-sectional view of a modified example of the configuration of the mixing device shown in FIG. 2.

FIG. 9 is a schematic cross-sectional view of another modified example of the configuration of the mixing device shown in FIG. 2.

FIG. 10 is a schematic cross-sectional view of still another modified example of the configuration of the mixing device shown in FIG. 2.

FIG. 11 is a schematic cross-sectional view of still another modified example of the configuration of the mixing device shown in FIG. 1.

FIG. 12 is a perspective view of a configuration of a mixing device and a multi-well plate according to a fifth embodiment of the present invention.

FIG. 13 is a cross-sectional view of a configuration of the mixing device shown in FIG. 12.

FIG. 14 is a cross-sectional view of the configuration of the mixing device and the multi-well plate shown in FIG. 12.

FIG. 15 is a perspective view of a configuration of a mixing unit of the mixing device shown in FIG. 12.

FIG. 16 is a perspective view of a partial configuration of the mixing unit of the mixing device shown in FIG. 12.

FIG. 17 is a cross-sectional view of a motor surrounding structure of the mixing device shown in FIG. 12.

FIG. 18 is a cross-sectional view of a motor surrounding structure of the mixing device shown in FIG. 12.

FIG. 19 is a graph showing a method of controlling a motor of the mixing device according to the present invention.

FIG. 20 is a perspective view of a configuration of a mixing device and a multi-well plate according to a sixth embodiment of the present invention.

FIG. 21 is a cross-sectional view of a configuration of the mixing device shown in FIG. 20.

FIG. 22 is a cross-sectional view of a configuration of the mixing device and the multi-well plate shown in FIG. 20.

### MODE(S) FOR CARRYING OUT THE INVENTION

Hereinafter, embodiments of the present invention will be described with reference to the drawings.

### First Embodiment

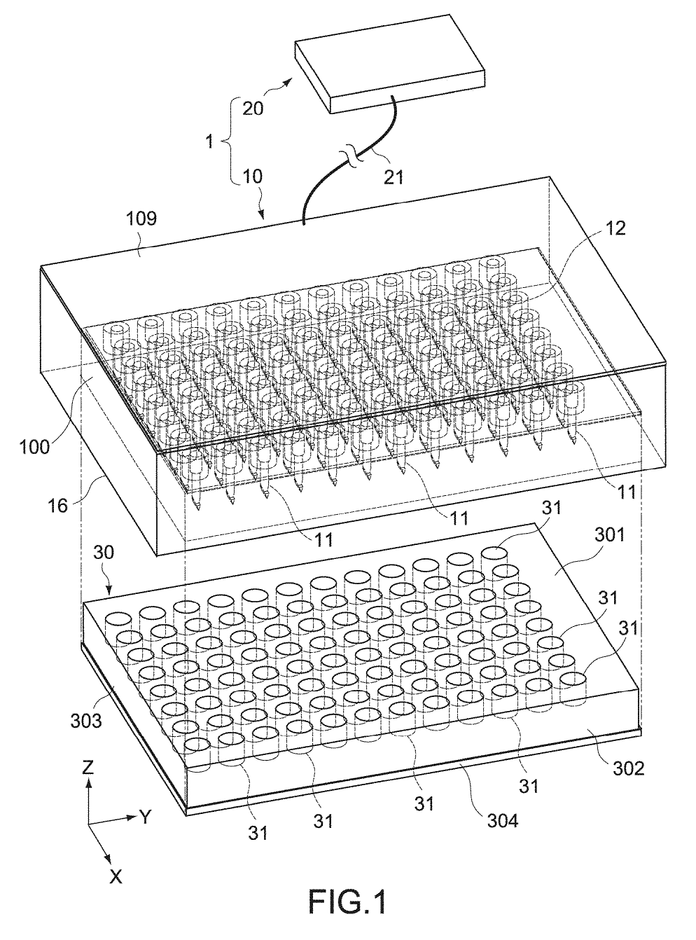

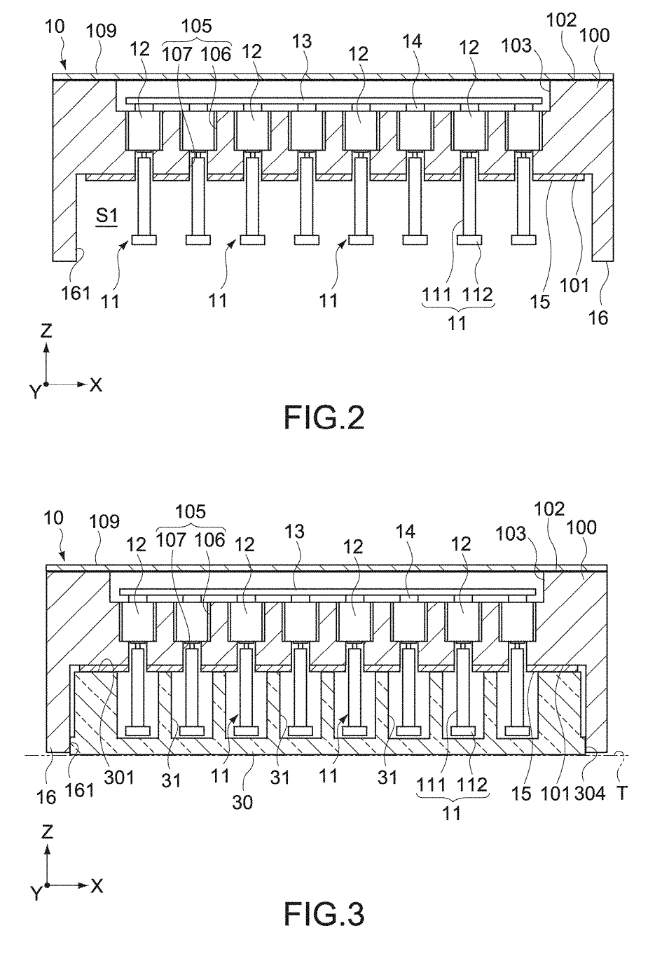

FIG. 1 is a perspective view of a mixing device according to a first embodiment of the present invention. FIG. 2 is a cross-sectional view of the mixing device along an X-axis direction. FIG. 3 is a cross-sectional view of the mixing device along the X-axis direction in a state of being attached to a multi-well plate.

It should be noted that in each figure the X- and Y-axis directions represent horizontal directions orthogonal to each other, and a Z-axis direction represents a height direction orthogonal to those directions.

[Overall Configuration]

A mixing device 1 of this embodiment includes a mixing unit 10 and a controller 20.

The mixing unit 10 is configured to be attachable to a multi-well plate 30. The mixing unit 10 includes a plurality of stirrers 11 for mixing a solution housed in each of wells 31 of the multi-well plate 30.

In this embodiment, the mixing unit 10 includes the plurality of stirrers 11 corresponding to the wells of the multi-well plate 30, but the mixing unit 10 is not limited thereto. The mixing unit 10 may include at least one stirrer.

The controller 20 is for controlling drive of the mixing unit 10 and is typically constituted of a computer including a CPU (Central Processing Unit), a memory (ROM (Read Only Memory), and a RAM (Random Access Memory)). The controller 20 may be constituted of a general-purpose computer or a dedicated computer.

The controller 20 is electrically connected to the mixing unit 10 and is configured so as to individually or commonly control rotations of motors that drive the stirrers 11. In this embodiment, the controller 20 is electrically connected to the mixing unit 10 via a wiring member 21, but the controller 20 is not limited thereto. For example, the controller 20 may be electrically connected to the mixing unit 10 wirelessly.

The multi-well plate 30 is constituted of a substantially rectangular plate-like member having an upper surface 301 on which the plurality of wells 31 are formed in a matrix, long-side side surfaces 302, and short-side side surfaces 303. The multi-well plate 30 is typically constituted of an injection-molded body made of a synthetic resin material having translucency.

The plurality of wells 31 are arranged in a matrix at predetermined intervals. In the example of the figure, eight wells 31 arrayed in a short-side direction (the X-axis direction) are arranged by twelve rows in a long-side direction (the Y-axis direction), so that a total of 96 wells are formed. An arrangement interval for the wells 31 is approximately 9 mm. It should be noted that the number of wells is not limited to this example and may be 6, 24, 384, 1536, or the like.

For the multi-well plate 30, commercially available general-purpose products are typically used. For example, “Nunc 96 micro-well plate” manufactured by Thermo Fisher Sceintific K.K is applicable.

[Mixing Unit]

Hereinafter, details of the mixing unit 10 will be described with reference to FIGS. 2 and 3.

The mixing unit 10 includes a casing 100, the plurality of stirrers 11, a plurality of motors 12, and a mounting portion 16.

The casing 100 is made of, for example, a metal material such as an aluminum alloy. The casing 100 is formed into a substantially rectangular plate shape, and one surface thereof is formed as a main surface portion 101 that faces the upper surface 301 of the multi-well plate 30. The main surface portion 101 is formed in a size capable of covering the upper surface 301 of the multi-well plate 30.

A concave portion 103 is formed on an upper surface portion 102 of the casing 100. The concave portion 103 houses a circuit substrate 13 that drives the plurality of motors 12. The upper surface portion 102 corresponds to a surface on the opposite side of the main surface portion 101. The concave portion 103 is covered with a cover 109 attached to the upper surface portion 102 of the casing 100.

The mounting portion 16 is integrally provided to the casing 100 as will be described later, and has a space portion S1 configured to be capable of housing the multi-well plate 30. As shown in FIG. 3, the mounting portion 16 is constituted of a peripheral wall hanging from a circumference of the main surface portion 101 toward the outer circumference of the multi-well plate 30, and forms the space portion S1 in the inside thereof. The height of the peripheral wall is set to a height at which the bottom portion of the peripheral wall does not come into contact with a work table T (see FIG. 3) when the casing 100 is placed on the upper surface 301 of the multi-well plate 30.

The plurality of stirrers 11 are disposed in a matrix in the casing 100 so as to correspond to all the wells 31 of the multi-well plate 30 housed in the space portion S1. The plurality of stirrers 11 protrude from the main surface portion 101 toward the multi-well plate 30 and are disposed inside the respective wells 31. The plurality of stirrers 11 have the same configuration and are respectively coupled to drive shafts of the plurality of motors 12 disposed in the casing 100.

In the mixing unit 10, the arrangement intervals of the stirrers 11 and the motors 12, the shape of the space portion S1, and the like are optimized depending on a type of a multi-well plate to be used (or the number of wells).

As shown in FIG. 2, in the casing 100, a plurality of stepped holes 105 that couple the concave portion 103 and the space portion S1 to each other are formed along the Z-axis direction. The plurality of stepped holes 105 are arranged in a matrix on the bottom surface of the concave portion 103. Each stepped hole 105 includes a large diameter portion 106 and a small diameter portion 107.

The large diameter portion 106 is located on the concave portion 103 side and formed in a size capable of housing the motor 12. The small diameter portion 107 is located on the space portion S1 side and formed in a size capable of housing the stirrer 11. The small diameter portion 107 is formed to be concentric with the large diameter portion 106. Each motor 12 is fixed to a step portion between the large diameter portion 106 and the small diameter portion 107.

The motor 12 configures a drive portion that rotates the stirrer 11 about its axis. The number of rotations of the motor 12 is not particularly limited. In this embodiment, the number of rotations of the motor 12 can be set in the range of 1 rpm to 6000 rpm, and a motor with ±2% or less of accuracy in number of rotations is used. This can cope with both of low-speed mixing and high-speed mixing and also can achieve highly accurate control of the number of rotations of the stirrers 11.

The motor 12 is constituted of a stepping motor that is driven by a pulse signal, but is not limited thereto. For example, a motor capable of highly accurately controlling the number of rotations, such as a synchronous motor or a brushless DC motor, is applicable. The size of the motor 12 is also not particularly limited, and a motor with a diameter of 6 mm or less is used, for example.

Each motor 12 is electrically connected to the circuit substrate 13 via a flexible wiring substrate 14. The circuit substrate 13 is electrically connected to the controller 20 via the wiring member 21. The drive of each motor 12 is configured to be individually controllable by the controller 20. Each motor 12 is driven by the same number of rotations (rotational speed) in the same rotational direction, but is not limited thereto. The rotational direction and the number of rotations can be made different for each of the motors. Further, all the motors 12 may be simultaneously activated or some of the motors 12 may be selectively activated.

Heat generated when the motors 12 are driven is discharged to the outside via the casing 100 made of metal. With this configuration, heat transfer to the multi-well plate 30 can be suppressed, and evaporation of a solution in the wells 31, transformation thereof due to heat, and the like can be suppressed.

The stirrer 11 includes a shaft portion 111 and a paddle portion 112. The shaft portion 111 is coupled to the drive shaft of the motor 12. The paddle portion 112 is formed at the tip of the shaft portion 111. The shape of the paddle portion 112 or the number thereof is not particularly limited, and various modes in which a desired function of mixing the solution is obtained by rotation about the axis of the shaft portion 111 can be employed.

As shown in FIG. 3, the stirrers 11 are disposed inside the respective wells 31 in a state where the multi-well plate 30 is housed in the space portion S1. Typically, each stirrer 11 is disposed on the central axis of each well 31. The height of the stirrer 11 from the bottom portion of the well 31 is not particularly limited and appropriately set in accordance with the size of the well 31, the amount of solution, a type, and the like. Typically, the height of the stirrer 11 is set to a height at which the tip of the stirrer 11 does not come into contact with the bottom portion of the well 31.

The mixing unit 10 further includes a sheet member 15 provided to the main surface portion 101. The sheet member 15 is configured so as to elastically come into contact with the upper surface of the multi-well plate 30 housed in a housing portion 104.

Providing the sheet member 15 is particularly effective in a case where a solution to be mixed is a volatile solution and can effectively prevent the solution from being evaporated due to a long-time mixing operation. A constituent material of the sheet member 15 is not particularly limited if the constituent material has heat resistance and chemical resistance and can elastically come into contact with the upper surface 301 of the multi-well plate 30. The constituent material is typically a silicone rubber.

The sheet member 15 is bonded to the main surface portion 101 of the casing 100 via an adhesive layer or the like. It is favorable that the sheet member 15 is detachably attached to the main surface portion 101. With this configuration, the sheet member 15 can be easily replaced, for example.

The mixing unit 10 of this embodiment includes the mounting portion 16 that is mounted to the multi-well plate 30 to position the casing 100 on the multi-well plate 30.

The mounting portion 16 is provided to the casing 100 and has an engaging surface 161 that comes into contact with the outer circumferential surface of the multi-well plate 30 housed in the space portion S1. As shown in FIG. 3, the engaging surface 161 is configured to be engageable with an outer circumferential surface of a convex portion 304 that is formed on the bottom portion of a side wall of the multi-well plate 30. The engaging surface 161 is typically formed of a flat surface (vertical surface), but is not limited thereto. The engaging surface 161 may be formed of a tapered surface or a curved surface.

The mounting portion 16 is mounted to the outer circumferential surface of the multi-well plate 30, so that the casing 100 is positioned with respect to the multi-well plate 30. From the perspective of ensuring the positioning accuracy, the mounting portion 16 is typically formed to be engageable with the four side surfaces (the entire circumference) of the multi-well plate 30, but is not limited thereto. The engaging position may be, for example, a part of the outer circumferential surface of the multi-well plate 30, for example, three side surfaces.

[Operation of Mixing Device]

Next, a typical operation of the mixing device 1 configured as described above will be described.

The mixing unit 10 is placed on the upper surface 301 of the multi-well plate 30, and thus the stirrers 11 are disposed inside the respective wells 31 of the multi-well plate 30. When the casing 100 is disposed on the upper surface 301 of the multi-well plate 30, the mounting portion 16 is engaged with the outer circumferential surface of the convex portion 304 of the multi-well plate 30 housed in the space portion S1. With this configuration, the casing 100 is positioned with respect to the multi-well plate 30.

The controller 20 outputs a drive pulse signal to the motor 12 and rotates the stirrer 11, which is disposed in the well 31 housing a solution to be mixed, by a predetermined number of rotations (for example, 3000 rpm). Typically, the controller 20 rotates each of the stirrers 11 by the same number of rotations, but may rotate the stirrers 11 by the number of rotations different for each of the wells. Furthermore, the controller 20 may simultaneously activate the motors or activate the motors in predetermined order.

At that time, the main surface portion 101 of the casing 100 comes into close contact with the upper surface 301 of the multi-well plate 30 via the sheet member 15. With this configuration, gaps between the adjacent wells 31 are shielded by the sheet member 15, and thus airborne droplets generated by mixing are prevented from being admixed in other wells 31. Further, the sheet member 15 improves airtightness of each of the wells 31. This suppresses evaporation of the solution in the wells 31.

In this embodiment, since the casing 100 is positioned with respect to the multi-well plate 30 by the mounting portion 16, each of the stirrers 11 is also disposed in each of the wells 31 with high position accuracy. With this configuration, the plurality of stirrers 11 can be collectively positioned with respect to the plurality of minute wells, and thus mixing treatment of the solution in each well can be made uniform.

Further, since the stirrers 11 are driven by the respective motors 12, the stirrers 11 can be rotated under optimal and appropriate driving conditions. Further, since each of the motors 12 is constituted of a stepping motor that can achieve an accurate number of rotations by a drive pulse, mixing accuracy and mixing efficiency for the solution in each of the wells 31 can be improved.

As described above, according to this embodiment, the mixing accuracy and mixing efficiency of each of the wells 31 can be considerably improved as compared with a horizontal vortex mixing method of circularly moving the entire plate in the horizontal direction. Further, according to this embodiment, the solution in each of the wells 31 can be individually mixed by the stirrer 11, and thus the mixing can be uniformly performed irrespective of the positions of the wells 31. Therefore, in a test method such as ELISA, a concentration of antibodies or antigens contained in a sample can be highly accurately detected or the quantity thereof can be determined.

Further, according to this embodiment, a mixing speed can be highly accurately controlled as compared with a method using a magnetic stirrer. Thus, it is possible to easily cope with various mixing conditions and to set different mixing conditions for each of the wells. Therefore, for example, also in a case where there are many dissolution test samples to be evaluated in the field of the pharmaceutical study or the like, efficient screening evaluation can be performed using the same multi-well plate.

### Second Embodiment

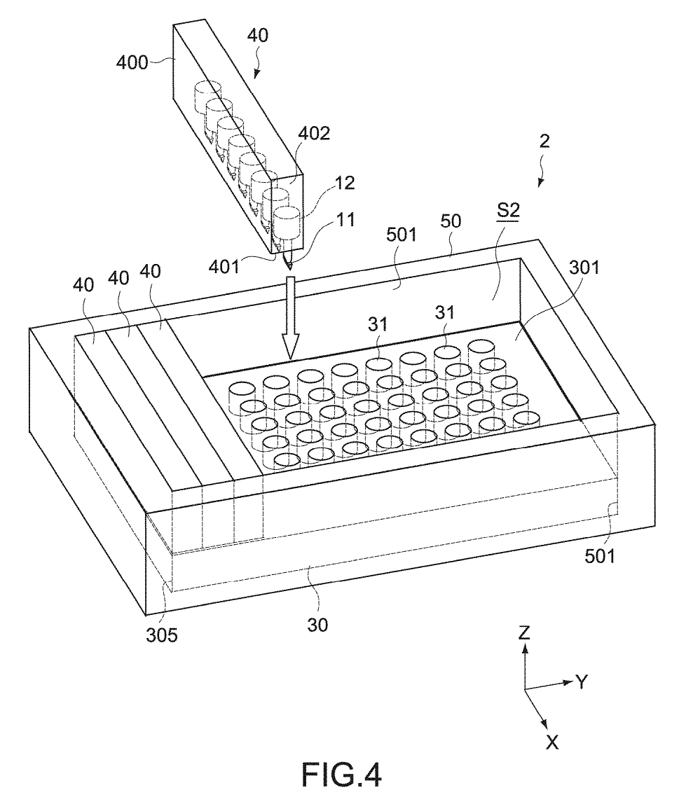

FIG. 4 is a perspective view of a configuration of a mixing device 2 according to a second embodiment of the present invention. Hereinafter, a configuration different from the first embodiment will be mainly described, and a configuration similar to the embodiment described above will be denoted by similar reference symbols and description thereof will be omitted or simplified.

The mixing device 2 of this embodiment includes a plurality of mixing units 40, a controller not shown in the figure, and a frame body 50.

The mixing units 40 are configured to be separated from one another for each of rows of wells 31 arranged in the Y-axis direction of a multi-well plate 30. Each of the mixing units 40 includes a plurality of (eight) stirrers 11 corresponding to eight wells 31 belonging to each row, a plurality of motors 12 that drive those stirrers 11, a circuit substrate (not shown) including drive circuits of the respective motors 12, and the like.

It should be noted that the mixing unit 40 is not limited to the configuration including the stirrers 11 corresponding to the number of wells in one row, and may be configured to include the stirrers 11 corresponding to the number of wells in two or more rows.

The mixing unit 40 includes a casing 400 that houses the plurality of stirrers 11 and the motors 12. The casing 400 has a rectangular parallelepiped shape and is made of a metal material such as an aluminum alloy. The casing 400 includes a main surface portion 401 placed on an upper surface 301 of the multi-well plate 30, and two side surfaces 402 that face each other in the X-axis direction.

The frame body 50 is configured to be separable from each of the mixing units 40 and has a rectangular frame shape having a space portion S2 therein. The space portion S2 is capable of housing the multi-well plate 30. The frame body 50 has an inner circumferential surface 501 that can be engaged (come into contact) with outer circumferential portions of the mixing units 40, which include the side surfaces 402, and with an outer circumferential portion 305 of the multi-well plate 30.

In this embodiment, the frame body 50 is mounted to the multi-well plate 30 and thus functions as a mounting portion that positions the casings 400 of the mixing units 40 with respect to the multi-well plate 30. In other words, in this embodiment, the mixing units 40 are mounted to the frame body 50 housing the multi-well plate 30 in the space portion S2, and thus the mixing units 40 are positioned with respect to the multi-well plate 30. Simultaneously, the stirrers 11 are highly accurately positioned with respect to the predetermined wells 31.

In the mixing device 2 of this embodiment, the motors 12 of the mixing units 40 are drive-controlled by the controller not shown in the figure. The controller 20 is configured to be electrically connectable with each of the mixing units 40 via the frame body 50, for example. In this case, the inner circumferential surface 501 of the frame body 50 and the outer circumferential portions (for example, the side surfaces 402) of the mixing units 40 may be provided with contact points that can be electrically connected to one another. Alternatively, the controller 20 may be electrically connected directly to each of the mixing units 40.

Also in the mixing device 2 of this embodiment configured as described above, an operational effect similar to that of the first embodiment described above can be obtained. According to this embodiment, the mixing units 40 are configured to be mountable to the wells 31 of the multi-well plate 30 on a row-by-row basis, and thus desired mixing treatment can be performed on a solution housed in not only all of the wells 31 but also some of the wells 31.

### Third Embodiment

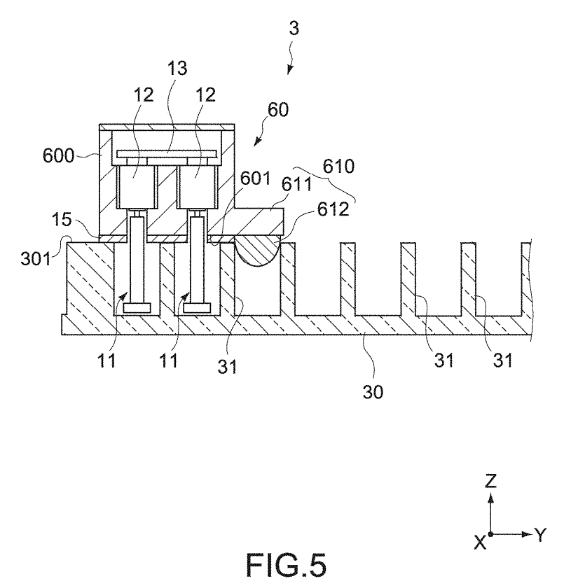

FIG. 5 is a cross-sectional view of a main part showing a configuration of a mixing device 3 according to a third embodiment of the present invention. Hereinafter, a configuration different from the first embodiment will be mainly described, and configurations similar to the embodiments described above will be denoted by similar reference symbols and description thereof will be omitted or simplified.

The mixing device 3 of this embodiment includes a mixing unit 60 and a controller not shown in the figure.

The mixing unit 60 includes a casing 600 that is formed in a size capable of covering the upper surfaces of wells 31 corresponding to two rows of the wells 31 arranged in the Y-axis direction of a multi-well plate 30. In the casing 600, a plurality of (16) stirrers 11 disposed to correspond to the wells 31 corresponding to the two rows, a plurality of motors 12 that drive those stirrers 11, and the like are disposed.

It should be noted that the mixing unit 60 is not limited to the configuration including the stirrers 11 corresponding to the number of wells in the two rows, and may be configured to include the stirrers 11 corresponding to the number of wells in one row or three or more rows.

The casing 600 has a schematically rectangular parallelepiped shape and is made of a metal material such as an aluminum alloy. The casing 600 includes a main surface portion 601 placed on an upper surface 301 of the multi-well plate 30. A sheet member 15 that can elastically come into close contact with the upper surface of the multi-well plate 30 is attached to the main surface portion 601.

The mixing unit 60 further includes a mounting portion 610. The mounting portion 610 includes a base portion 611 integrally formed with the casing 600, and a plurality of engaging protrusions 612 formed on the lower surface of the base portion 611.

The base portion 611 is provided to extend in the Y-axis direction from the lower end of the casing 600 over the length corresponding to one row of the wells. The thickness of the base portion 611 is not particularly limited, and the base portion 611 may be formed in a thickness (height) equivalent to that of the casing 600.

The plurality of engaging protrusions 612 are disposed to correspond to the respective wells 31 located just below the base portion 611 and are configured to be engageable with opening portions of the wells 31. In this embodiment, each of the engaging protrusions 612 is formed in a substantially hemisphere shape, but is not limited thereto. Each of the engaging protrusions 612 may be formed in a circular shape, a rectangular cylinder shape, or another geometric shape.

Also in the mixing device 3 of this embodiment configured as described above, an operational effect similar to that of the first embodiment described above can be obtained. According to this embodiment, as in the second embodiment, the mixing unit 60 is configured to be mountable to the wells 31 of the multi-well plate 30 in predetermined units of row, and thus desired mixing treatment can be performed on a solution housed in not only all of the wells 31 but also some of the wells 31.

Further, in this embodiment, the mounting portion 610 includes the plurality of engaging protrusions 612 that are configured to be engaged with the plurality of wells belonging to a row different from the rows in which the stirrers 11 are disposed, and thus downsizing and weight saving of the mixing unit 60 can be achieved.

It should be noted that the well row with which the engaging protrusions 612 are engaged is not limited to the row adjacent to the well rows in which the stirrers 11 are disposed. Further, the number of engaging protrusions 612 does not necessarily correspond to the number of wells (eight) in the row, and the engaging protrusions 612 only need to be configured to be engageable with at least two wells.

### Fourth Embodiment

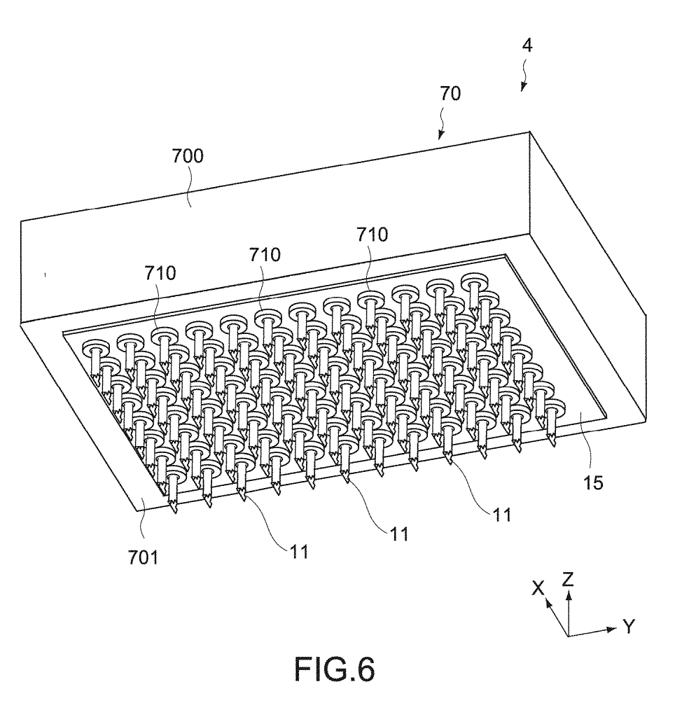

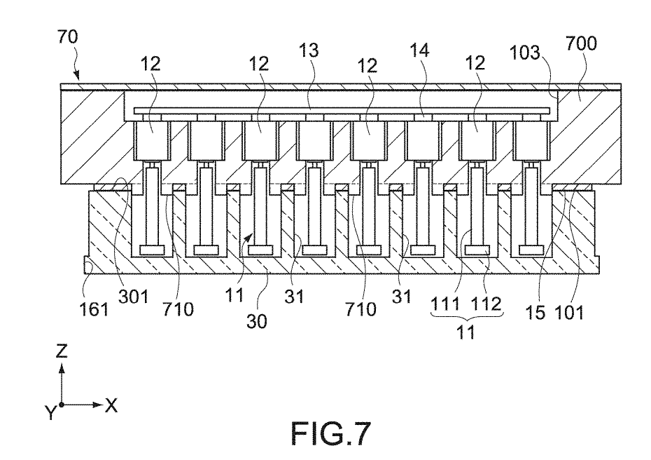

FIGS. 6 and 7 each show a configuration of a mixing device according to a fourth embodiment of the present invention. FIG. 6 is a perspective view, and FIG. 7 is a side cross-sectional view. Hereinafter, a configuration different from the first embodiment will be mainly described, and configurations similar to the embodiments described above will be denoted by similar reference symbols and description thereof will be omitted or simplified.

A mixing device 4 of this embodiment includes a mixing unit 70 and a controller not shown in the figures.

The mixing unit 70 includes a casing 700 that is formed in a size capable of covering an upper surface 301 of a multi-well plate 30, similarly to the first embodiment. In the casing 700, a plurality of stirrers 11 disposed to correspond to wells 31 of the multi-well plate 30, a plurality of motors 12 that drive those stirrers 11, and the like are disposed.

The casing 700 has a schematically rectangular parallelepiped shape and is made of a metal material such as an aluminum alloy. The casing 700 includes a main surface portion 701 placed on the upper surface 301 of the multi-well plate 30. A sheet member 15 that can elastically come into close contact with the upper surface of the multi-well plate 30 is attached to the main surface portion 701.

The mixing unit 70 further includes a plurality of mounting portions 710. Each of the mounting portions 710 is constituted of an annular convex portion integrally formed with the main surface portion 701 of the casing 700 and is formed so as to protrude from the main surface portion 701. The plurality of mounting portions 710 are fit into the respective wells 31 in a state where the sheet member 15 is in close contact with the upper surface 301 of the multi-well plate 30. With this configuration, the casing 700 is positioned with respect to the multi-well plate 30, and the stirrers 11 are disposed with respect to the respective wells 31 with high position accuracy.

Also in the mixing device 4 of this embodiment configured as described above, an operational effect similar to that of the first embodiment described above can be obtained.

The embodiments of the present invention have been described above, but the present invention is not limited to the embodiments described above and can be variously modified without departing from the gist of the present invention as a matter of course.

For example, in the above embodiments, the multi-well plate 30 in which the number of wells is 96 is used, but the present invention is not limited thereto. Another multi-well plate having a different number of wells may be used. In this case, an arrangement pitch of the stirrers, the size of the mounting portion, and the like are optimized in accordance with the outer shape of the multi-well plate and an arrangement pitch of the wells.

For further improvement in accuracy of the number of rotations of the stirrers 11, the number of rotations of each stirrer 11 may be configured to be capable of being monitored by the controller 20. In this case, for example, the casing is provided with a detection portion such as an encoder for detecting the number of rotations of the stirrers 11, and the controller 20 is configured to control the stirrers 11 by a predetermined number of rotations on the basis of an output of the detection portion.

Further, in the above embodiments, in order to suppress the evaporation of the solution in the wells 31, the sheet member 15 is provided to the main surface portion of the casing. However, instead of this or in addition to this, an inner pressure of each well may be kept to a predetermined pressure to suppress the evaporation of the solution.

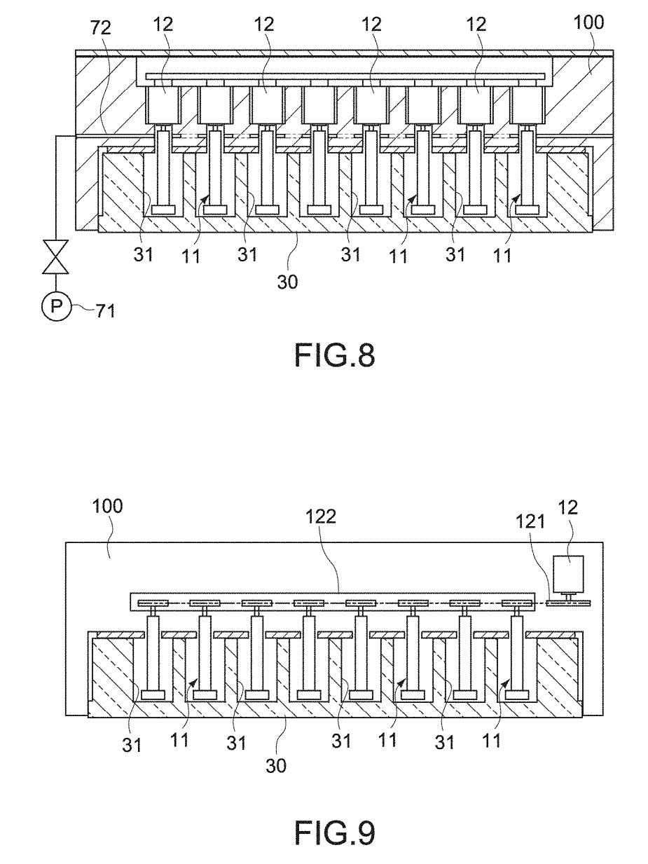

For example, a mixing unit shown in FIG. 8 includes a pressurizing pump 71 and a passage hole 72 connected to a discharge opening of the pressurizing pump 71. The passage hole 72 is configured to be capable of introducing gas discharged from the pressurizing pump 71 into wells 31 in which stirrers 11 are disposed. For example, the passage hole 72 is formed in the casing 100 in a grid pattern so as to communicate with the small diameter portions 107 of the stepped holes. The pressurizing pump 71 discharges gas of a pressure corresponding to, for example, a saturation water vapor pressure in the wells. With this configuration, it is possible to suppress the evaporation of the solution in the wells. The gas discharged from the pressurizing pump may be air or inert gas of argon or the like.

Further, in the above embodiments, the plurality of motors 12 disposed to correspond to the plurality of stirrers 11 are used as the drive portion, but the plurality of stirrers 11 may be configured to be rotated by a single motor.

For example, a mixing unit shown in FIG. 9 includes a gear row 122 that transmits a rotational drive force of the single motor 12 to each of the stirrers 11. The gear row 122 is connected to a main gear 121 coupled to the motor 12. The gear row 122 includes a plurality of gears that transmit rotation of the main gear 121 to each of the stirrers 11.

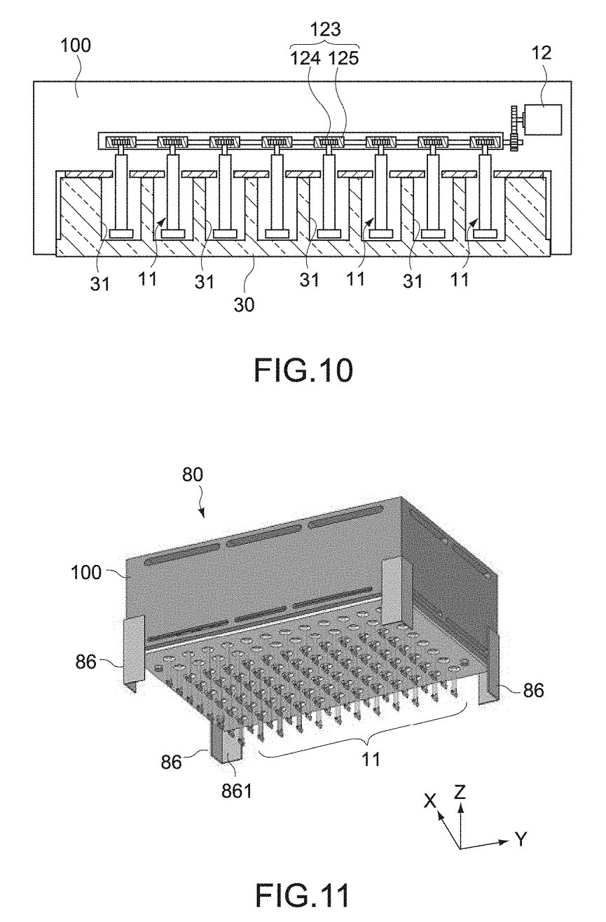

Meanwhile, a mixing unit shown in FIG. 10 includes a gear unit 123 that transmits a rotational drive force of the single motor 12 to each of the stirrers 11. The gear unit 123 includes a plurality of pinion gears 124 fixed to base ends of the respective stirrers 11 and a plurality of worm gears 125 that transmit a drive force of the motor 12 to those pinion gears 124. According to this configuration, a rotational speed of the stirrer 11 can be adjusted using a gear ratio of the worm gears 125, and thus for example, low-speed and smoother mixing can be stably performed.

Furthermore, in the first embodiment described above, the mounting portion 16 of the mixing unit 20 is configured so as to be engaged with the entire outer circumferential surface of the multi-well plate 30 via the engaging surface 161. However, instead of this, the mounting portion 16 may be configured so as to be engaged with a part of the outer circumferential surface of the multi-well plate 30. Also with this configuration, an operational effect similar to that of the first embodiment can be obtained. For example, FIG. 11 shows a mixing unit 80 including four mounting portions 86 that partially come into contact with the outer circumferential surface of the multi-well plate 30 at the four corners only. Each of the mounting portions 86 is constituted of a curved member that is bent by approximately 90° about the Z axis, and the inner surfaces of the mounting portions 86 are configured as engaging surfaces 861 that are engaged (come into contact) with the outer circumferential surface of the multi-well plate 30 at the four corners.

### Fifth Embodiment

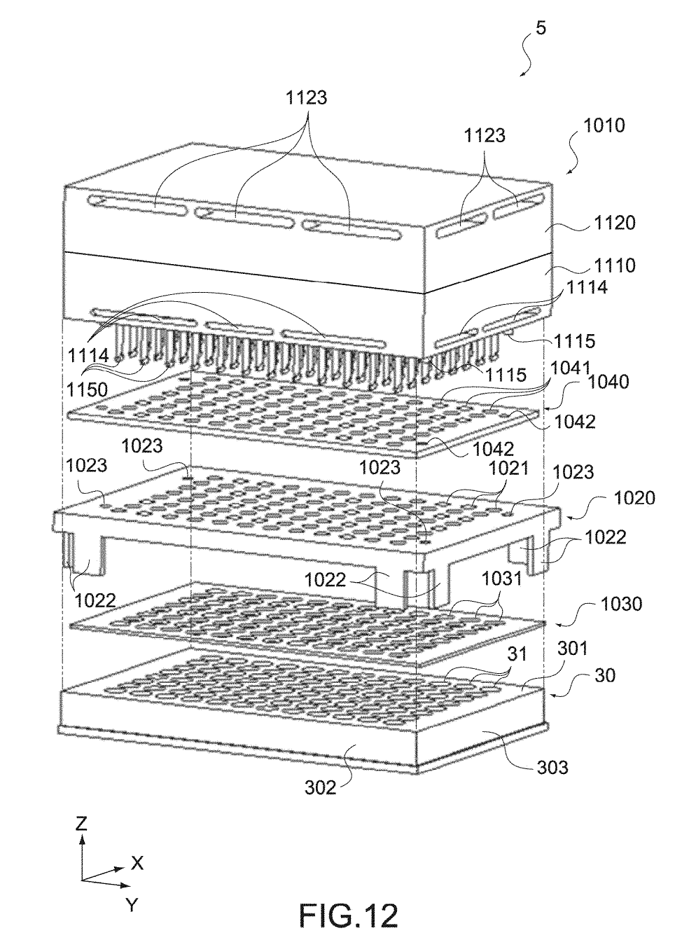

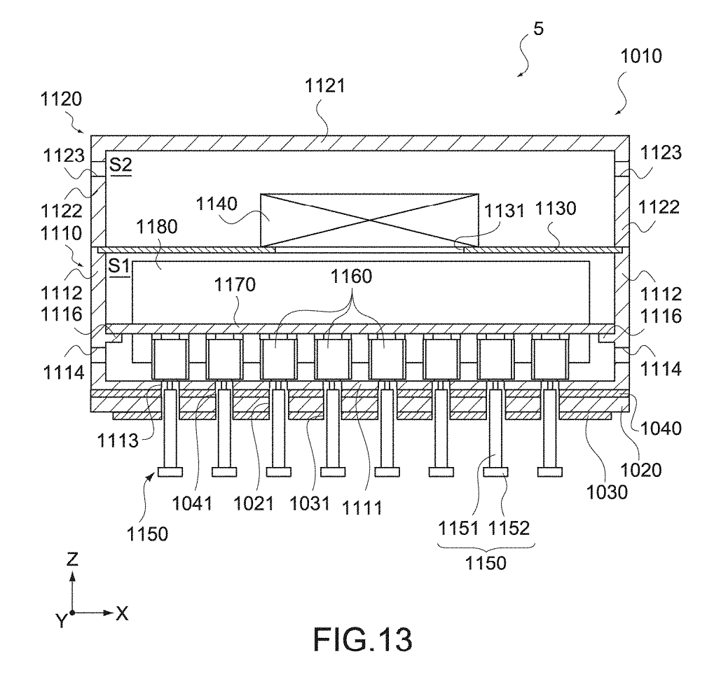

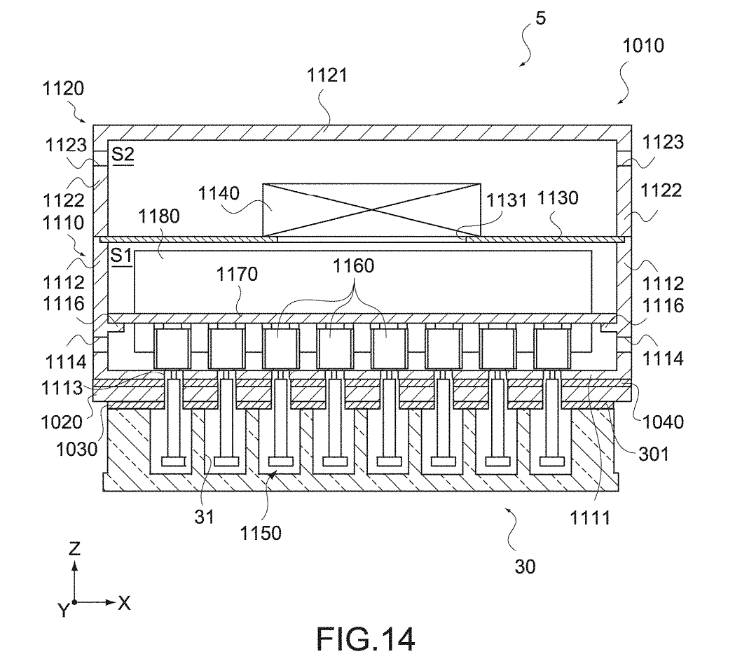

FIG. 12 is an exploded perspective view of a mixing device 5 and a multi-well plate 30 according to a fifth embodiment of the present invention. FIG. 13 is a cross-sectional view of the mixing device 5 along the X-axis direction. FIG. 14 is a cross-sectional view of the mixing device 5 along the X-axis direction in a state of being attached to the multi-well plate 30. Hereinafter, a configuration different from the first embodiment will be mainly described, and configurations similar to the embodiments described above will be denoted by similar reference symbols and description thereof will be omitted or simplified.

It should be noted that in each figure the X- and Y-axis directions represent horizontal directions orthogonal to each other, and the Z-axis direction represents a height direction orthogonal to those directions.

[Overall Configuration]

As shown in FIG. 12, the mixing device 5 includes a mixing unit 1010, an attachment 1020, a first sheet member 1030, and a second sheet member 1040. Further, the mixing device 5 includes a controller that is not shown in the figures as in the first embodiment.

The mixing unit 1010 is configured to be attachable to the multi-well plate 30 via the attachment 1020. The mixing unit 1010 includes a plurality of stirrers 1150 for mixing a solution housed in wells 31 of the multi-well plate 30.

In this embodiment, the mixing unit 1010 includes the plurality of stirrers 1150 corresponding to the wells of the multi-well plate 30, but the mixing unit 1010 is not limited thereto. The mixing unit 1010 may include at least one stirrer.

The attachment 1020 includes through-holes 1021, abutment portions 1022, and positioning holes 1023. The attachment 1020 can be a plate-like member having a size equivalent to the size of the multi-well plate 30.

The through-holes 1021 are holes that penetrate the front and rear surfaces of the attachment 1020 as shown in FIG. 13, and are provided to the respective wells of the multi-well plate 30 one by one. Each of the through-holes 1021 has a hole diameter that is smaller than the well 31 and larger than the stirrer 1150. Each stirrer 1150 is inserted into each through-hole 1021.

As shown in FIG. 12, the abutment portions 1022 are parts hanging from a circumference of the attachment 1020 toward an outer circumferential surface of the multi-well plate 30. The abutment portions 1022 abut on the outer circumferential surface of the multi-well plate 30, and thus position the attachment 1020 with respect to the multi-well plate 30.

Specifically, the abutment portion 1022 can include a part that abuts on a side surface 302 of the multi-well plate 30 and a part that abuts on a side surface 303 thereof. It should be noted that a specific shape of the abutment portion 1022 is not limited to that shown in FIG. 12 as long as the attachment 1020 can be positioned with respect to the multi-well plate 30.

The positioning holes 1023 are holes into which positioning pins 1115 of the mixing unit 1010 are inserted. The number of the positioning holes 1023 or the shape thereof is not particularly limited, and four positioning holes can be provided on the circumference of the attachment 1020 as shown in FIG. 12.

A constituent material of the attachment 1020 is not particularly limited and can be made of a synthetic resin having heat resistance and chemical resistance, or the like.

The first sheet member 1030 is a sheet-like member made of an elastic material, which is disposed between the attachment 1020 and the multi-well plate 30 and includes through-holes 1031. The through-holes 1031 are holes that penetrate the front and rear surfaces of the first sheet member 1030 as shown in FIG. 13, and are provided to the respective wells 31 of the multi-well plate 30 one by one.

A constituent material of the first sheet member 1030 is not particularly limited if the constituent material is a material that has heat resistance and chemical resistance and can elastically come into contact with the upper surface 301 of the multi-well plate 30 and the attachment 1020, and is typically a silicone rubber.

The second sheet member 1040 is a sheet-like member made of an elastic material, which is disposed between the attachment 1020 and the mixing unit 1010 and includes through-holes 1041. The through-holes 1041 are holes that penetrate the front and rear surfaces of the second sheet member 1040 as shown in FIG. 13, and are provided to the respective wells of the multi-well plate 30 one by one.

Further, as shown in FIG. 12, the second sheet member 1040 is provided with positioning holes 1042. The positioning holes 1042 are holes which penetrate the front and rear surfaces of the first sheet member 1040 and into which the positioning pins 1115 of the mixing unit 1010 are inserted. The number of the positioning holes or the shape thereof is not particularly limited, and four positioning holes can be provided on the circumference portion of the second sheet member 1040 as shown in FIG. 12.

A constituent material of the second sheet member 1040 is not particularly limited if the constituent material is a material that has heat resistance and chemical resistance and can elastically come into contact with the mixing unit 1010 and the attachment 1020, and is typically a silicone rubber.

As in the first embodiment, the controller is for controlling drive of the mixing unit 1010. The controller is electrically connected to the mixing unit 1010 and is configured so as to individually or commonly control rotations of motors that drive the stirrers 1150.

[Mixing Unit]

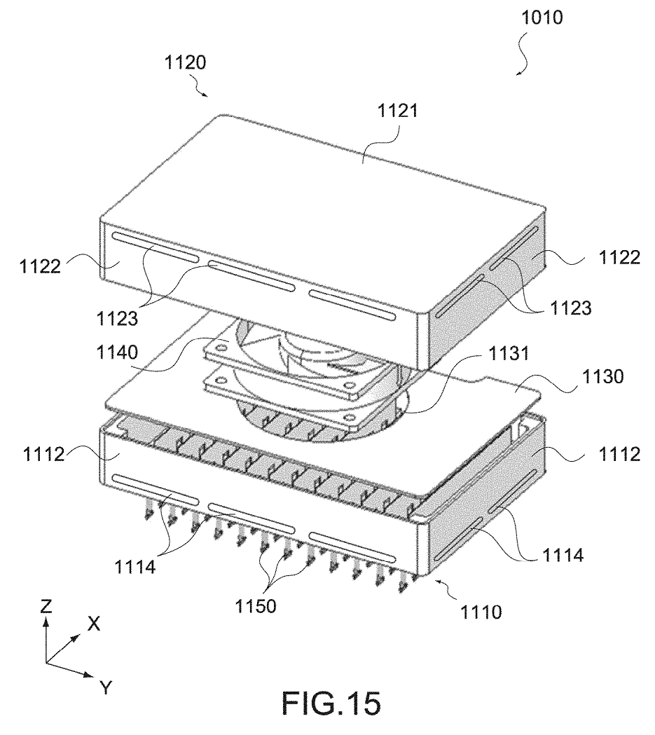

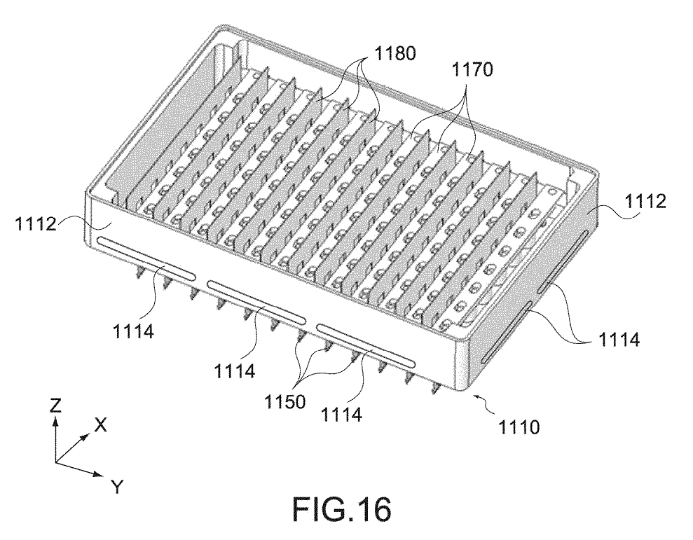

Details of the mixing unit 1010 will be described. FIG. 15 is an exploded perspective view of the mixing unit 1010. FIG. 16 is a perspective view of a partial configuration of the mixing unit 1010.

As shown in FIGS. 14 to 16, the mixing unit 1010 includes a first casing 1110, a second casing 1120, a fan mounting plate 1130, a fan 1140, stirrers 1150, motors 1160, motor retaining plates 1170, and 1180.

The first casing 1110 is made of, for example, a metal material such as an aluminum alloy. The first casing 1110 includes a main surface portion 1111 that faces the upper surface 301 of the multi-well plate 30 and a side-wall portion 1112 hanging from the main surface portion 1111.

The main surface portion 1111 includes a plurality of through-holes 1113. The through-holes 1113 penetrate the main surface portion 1111. Each stirrer 1150 is inserted into each through-hole 1113.

Further, the first casing 1110 includes vents 1114. The vents 1114 penetrate the side-wall portion 1112 and cause the inside and the outside of the first casing 1110 to communicate with each other. The shape of the vent 1114 or the number thereof is not particularly limited.

As shown in FIG. 12, the positioning pins 1115 are provided on the circumference of the main surface portion 1111. The positioning pins 1115 protrude from the main surface portion 1111 and are inserted into the positioning holes 1042 of the second sheet member 1040 and the positioning holes 1023 of the attachment 1020. With this configuration, the mixing unit 1010 is positioned with respect to the attachment 1020 and positioned with respect to the multi-well plate 30 via the attachment 1020.

Further, as shown in FIG. 14, the side-wall portion 1112 includes a motor-retaining-plate support portion 1116. The motor-retaining-plate support portion 1116 protrudes from the side-wall portion 1112 in a housing space and is configured so as to be capable of placing the motor retaining plates 1170 thereon.

The second casing 1120 is made of, for example, a metal material such as an aluminum alloy. The second casing 1120 includes a flat plate-like main surface portion 1121 and a side-wall portion 1122 hanging from the main surface portion 1121.

Further, the second casing 1120 includes vents 1123. The vents 1123 penetrate the side-wall portion 1122 and cause the inside and the outside of the second casing 1120 to communicate with each other. The shape of the vent 1123 or the number thereof is not particularly limited.

The side-wall portion 1112 of the first casing 1110 and the side-wall portion 1122 of the second casing 1120 are joined to each other, and the first casing 1120 and the second casing 1120 form an internal space.

The fan mounting plate 1130 is sandwiched by the side-wall portion 1112 of the first casing 1120 and the side-wall portion 1122 of the second casing 1120 and supports the fan 1140. As shown in FIGS. 14 and 15, the fan mounting plate 1130 is provided with an opening 1131 that penetrates the fan mounting plate 1130. The shape of the opening 1131 or the number thereof is not particularly limited.

The internal space formed by the first casing 1110 and the second casing 1120 is partitioned by the fan mounting plate 1130. Hereinafter, as shown in FIG. 14, a space formed by the first casing 1110 and the fan mounting plate 1130 is assumed as an internal space S1, and a space formed by the second casing 1120 and the fan mounting plate 1130 is assumed as an internal space S2. The internal space S1 and the internal space S2 communicate with each other by the opening 1131 provided to the fan mounting plate 1130.

The fan 1140 is fixed to the fan mounting plate 1130 and faces the opening 1131. The fan 1140 only needs to have a configuration capable of generating an airflow and is, for example, a fan that can rotate a propeller with use of a built-in motor.

The stirrers 1150 are disposed in a matrix so as to correspond to all the wells 31 of the multi-well plate 30. The stirrers 1150 are inserted into the through-holes 1113, the through-holes 1041, the through-holes 1021, and the through-holes 1031 as shown in FIG. 13, and disposed inside the respective wells 31 as shown in FIG. 14. The stirrers 1150 have the same configuration and are connected to the respective motors 1160.

As shown in FIG. 13, each of the stirrers 1150 includes a shaft portion 1151 and a paddle portion 1152. The shaft portion 1151 is connected to the motor 1160. The paddle portion 1152 is formed at the tip of the shaft portion 1151. The shape of the paddle portion 1152 or the number thereof is not particularly limited, and various modes in which a desired function of mixing a solution is obtained by rotation about the axis of the shaft portion 1151 can be employed.

As shown in FIG. 14, the stirrers 1150 are disposed inside the respective wells 31 in a state where the mixing unit 1010 is attached to the multi-well plate 30. Typically, each stirrer 1150 is disposed on the central axis of each well 31. The height of the stirrer 1150 from the bottom portion of the well 31 is not particularly limited and appropriately set in accordance with the size of the well 31, the amount of solution, a type, and the like. Typically, the height of the stirrer 1150 is set to a height at which the tip of the stirrer 1150 does not come into contact with the bottom portion of the well 31.

The motor 1160 configures a drive portion that rotates the stirrer 1150 about its axis. The motor 1160 is constituted of a stepping motor that is driven by a pulse signal as in the first embodiment, but is not limited thereto. For example, a motor capable of highly accurately controlling the number of rotations, such as a synchronous motor or a brushless DC motor, is applicable.

Each motor 1160 is electrically connected to the circuit substrate 1180 and is configured to be individually controllable by the controller. Each motor 1160 is driven by the same number of rotations (rotational speed) in the same rotational direction, but is not limited thereto. The rotational direction and the number of rotations can be made different for each of the motors. Further, all the motors 1160 may be simultaneously activated or some of the motors 1160 may be selectively activated.

The motor retaining plates 1170 are supported by the motor-retaining-plate support portion 1116 by screwing or the like and fix the motors 1160. As shown in FIG. 16, the motor retaining plate 1170 is a plate-like member extending in the X direction, and the plurality of motor retaining plates 1170 are arranged in parallel with the Y direction.

Each of the motor retaining plates 1170 fixes the plurality of motors 1160 arranged in the X direction. The motors 1160 are fixed by the motor retaining plates 1170 by screwing or the like. It should be noted that the shape of the motor retaining plate 1170 or the arrangement thereof is not particularly limited, and the motor retaining plate 1170 only needs to be capable of fixing each of the motors 1160 to the first casing 1110.

The circuit substrate 1180 is connected to the controller and supplies a drive signal to each of the motors 1160. The circuit substrate 1180 is electrically connected to the plurality of motors 1160 arranged in the X direction, and the plurality of circuit substrates 1180 are arranged in parallel with the Y direction. The configuration of the circuit substrate 1180 is not particularly limited. The motors 1160 may be connected to individual circuit substrates or all the motors 1160 may be connected to one circuit substrate.

The circuit substrate 1180 may include a drive circuit of the motor 1160, but substantially performs only connection of the motor 1160. A drive circuit may be disposed outside the mixing unit 1010.

[Operation of Mixing Device]

Next, a typical operation of the mixing device 5 configured as described above will be described.

As shown in FIG. 12, the mixing unit 1010 is placed on the upper surface 301 of the multi-well plate 30 via the attachment 1020. With this configuration, the stirrers 1150 are disposed inside the respective wells 31 of the multi-well plate 30.

The abutment portions 1022 of the attachment 1020 abut on the multi-well plate 30, and the attachment 1020 is positioned with respect to the multi-well plate 30. Further, the positioning pins 1115 of the mixing unit 1010 are inserted into the positioning holes 1023 of the attachment 1020, and the mixing unit 1010 is positioned with respect to the attachment 1020. With this configuration, the mixing unit 1010 is positioned with respect to the multi-well plate 30.

As in the first embodiment, the controller outputs a drive pulse signal to the motor 1160 and rotates the stirrer 1150, which is disposed in the well 31 housing a solution to be mixed, by a predetermined number of rotations (for example, 3000 rpm). Typically, the controller rotates each of the stirrers 1150 by the same number of rotations, but may rotate the stirrers 1150 by the number of rotations different for each of the wells. Furthermore, the controller may simultaneously activate the motors or activate the motors in predetermined order.

At that time, as shown in FIG. 14, the first sheet member 1030 comes into close contact with the upper surface 301 of the multi-well plate 30 and the attachment 1020, and the second sheet member 1040 comes into close contact with the attachment 1020 and the main surface portion 1111 of the first casing 1110.

With this configuration, gaps between the adjacent wells 31 are shielded by the first sheet member 1030, the attachment 1020, and the second sheet member 1040, and airborne droplets generated by mixing are prevented from being admixed in other wells 31. Further, the first sheet member 1030 and the second sheet member 1040 improve airtightness of each of the wells 31. This suppresses evaporation of the solution in the wells 31.

In this embodiment, the mixing unit 1010 is positioned with respect to the multi-well plate 30 by the abutment portions 1022 of the attachment 1020 and the positioning pins 1115 of the mixing unit 1010. With this configuration, each of the stirrers 1150 is also disposed in each of the wells 31 with high position accuracy. Since the plurality of stirrers 1150 can be collectively positioned with respect to the plurality of minute wells, mixing treatment of the solution in each well can be made uniform.

Further, since the stirrers 1150 are driven by the respective motors 1160, each of the stirrers 1150 can be rotated under optimal and appropriate driving conditions. Further, since each of the motors 1160 is constituted of a stepping motor that can achieve an accurate number of rotations by a drive pulse, mixing accuracy and mixing efficiency for the solution in each of the wells 31 can be improved.

Furthermore, in this embodiment, the fan 1140 is driven by the controller, and an airflow flowing in the internal space S1 from the internal space S2 via the opening 1131 is generated. Further, the first casing 1110 and the second casing 1120 are respectively provided with the vents 1114 and the vents 1123. Therefore, air flows in the internal space S2 from the outside of the mixing unit 1010 via the vents 1123, flows in the internal space S1 by the fan 1140, and is then discharged from the internal space S1 to the outside of the mixing unit 1010 via the vents 1114.

With this configuration, an airflow that flows in from the outside of the mixing unit 1010 and flows out again to the outside of the mixing unit 1010 is generated around the motors 1160 housed in the internal space S1, and the motors 1160 are cooled by the airflow.

It should be noted that the orientation of the discharge of the fan 1140 may be opposite to the above. In this case, air flows in the internal space S1 from the outside of the mixing unit 1010 via the vents 1114, flows in the internal space S2 by the fan 1140, and is then discharged from the internal space S2 to the outside of the mixing unit 1010 via the vents 1114.

In such a manner, the heat generated when the motors 1160 are driven is cooled also by the airflow generated by the fan 1140, in addition to heat transfer to the first casing 1110 and the second casing 1120 that are made of metal. With this configuration, the heat transfer to the multi-well plate 30 can be suppressed, and evaporation, transformation due to the heat, or the like of the solution in the wells 31 can be suppressed.

It should be noted that the vents 1123 and the vents 1114 are not necessarily provided. For example, another opening may be provided at a position different from the opening 1131 of the fan mounting plate 1130. In this case, air flowing from the internal space S2 to the internal space S1 via the opening 1131 by the fan 1140 goes back to the internal space S2 via the other opening. In other words, an airflow circulating inside the mixing unit 1010 is generated, and the motors 1160 are cooled.

[Regarding Motor Surrounding Structure]

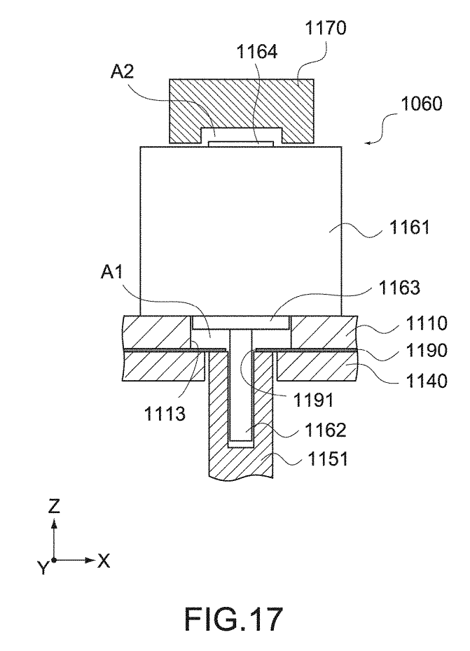

The motor 1160 described above can have the following surrounding structure. FIG. 17 is a cross-sectional view of a surrounding structure of the motor 1160. As shown in the figure, the motor 1160 includes a motor chassis 1161, a motor shaft 1162, a bearing 1163, and a bearing 1164.

The motor chassis 1161 stores a rotor and a stator of the motor. The motor shaft 1162 is connected to the rotor. The bearing 1163 and the bearing 1164 are fixed to the motor chassis 1161 and rotatably supports the motor shaft 1162. The shaft portion 1151 of the stirrer 1150 is connected to the motor shaft 1162.

A sealing 1190 is provided between the first casing 1110 and the second sheet member 1040. The sealing 1190 is made of an elastic material having heat resistance and chemical resistance, such as a silicone rubber, and has an opening 1191. The opening 1191 penetrates both sides of the sealing 1190 and has an opening diameter that is larger than the motor shaft 1162 and smaller than the shaft portion 1151.

The sealing 1190 isolates a space (around the shaft portion 1151) communicating with the liquid to be mixed and the bearing 1163 from each other and prevents airborne droplets and vapor of the liquid to be mixed from reaching the bearing 1163. While it is desirable to apply grease to the bearing 1163 and the bearing 1164, sealing by the sealing 1190 can prevent a lubricating grease from outflowing or degrading due to vapor of the liquid to be mixed or the like.

Further, the sealing 1190 seals the gap between the shaft portion 1151 and the second sheet member 1040, so that a grease holding space A1 is formed in a gap between the bearing 1163, the first casing 1110, and the sealing 1190. The grease holding space A1 is filled with the lubricating grease, and thus the bearing 1163 can be isolated from the space communicating with the liquid to be mixed.

Furthermore, a grease holding space A2 is formed between the bearing 1164 and the motor retaining plates 1170. The grease holding space A2 isolates the bearing 1164 from outside air, and thus the bearing 1164 can be prevented from being degraded. Further, the grease holding space A2 may be filled with the lubricating grease and the bearing 1164 may be isolated from outside air.

In such a manner, the bearing 1163 and the bearing 1164 are isolated from the liquid to be mixed or the outside air, and thus those bearings can be prevented from being degraded, and the useful life of the motor 1160 can be extended.

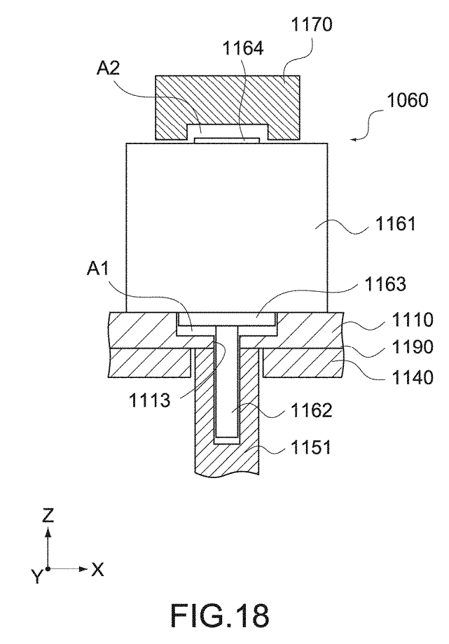

It should be noted that the first casing 1110 may be used instead of the sealing 1190. FIG. 18 is a cross-sectional view of a surrounding structure of the motor 1160 in this case. As shown in the figure, the through-hole 1113 provided to the first casing 1110 can have an opening diameter that is larger than the motor shaft 1162 and smaller than the shaft portion 1151. In this structure, the first casing 1110 seals the gap between the shaft portion 1151 and the second sheet member 1040, and the grease holding space A1 is thus formed, so that the bearing 1163 can be isolated from the space communicating with the liquid to be mixed.

[Regarding Motor Control]

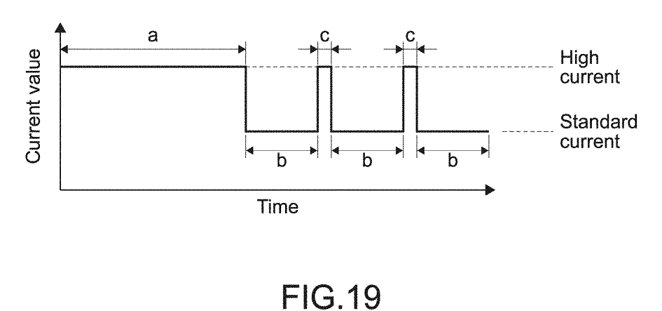

For a drive current of the motor 1160, it is desirable to generate an optimal torque at the time of driving and also suppress heat generation as much as possible. FIG. 19 is a graph showing an example of controlling the drive current of the motor 1160 by the controller. As shown in the figure, it is desirable for the controller to switch the drive current to a high current significantly larger than a standard current value and change a duty cycle thereof at the time of activation and the time of normal operation.

For example, “a” of FIG. 19 represents a period of time immediately after the motor 1160 starts to rotate. For example, a high current is caused to flow for approximately ten seconds to generate a high torque at the time of the rotation start, and thus the stirrer 1150 can be reliably rotated.

Further, the controller can set the drive current to a standard current after the activation (“b” in the figure) and set the drive current to a high current at constant intervals (“c” in the figure). Setting the drive current to a standard current reduces the torque generated by the motor 1160 but can prevent heat generation of the motor 1160.

Further, setting the drive current to a high current at constant intervals can increase the torque generated by the motor 1160 and immediately restore rotation even if the motor causes a loss of synchronization due to contact of the stirrer 1150 to a solid material, or the like.

A pulse width can be set as follows, for example, “b” is 999 msec, and “c” is 1 msec, but depending on circumstances, the duty cycle can be adequately set. The value of the high current is favorably approximately twice as large as the standard current, but the value is not limited thereto and can be adequately selected depending on circumstances. It should be noted that the current control as described above can also be performed similarly in other embodiments of the present invention.

### Sixth Embodiment

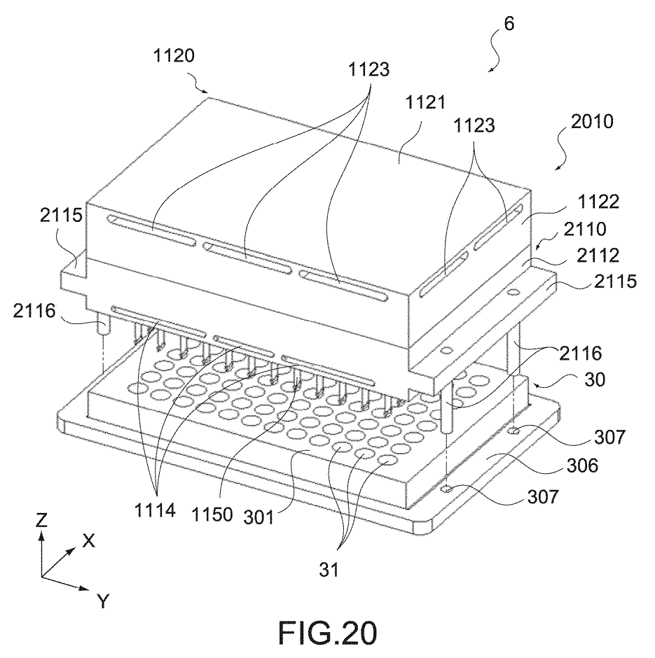

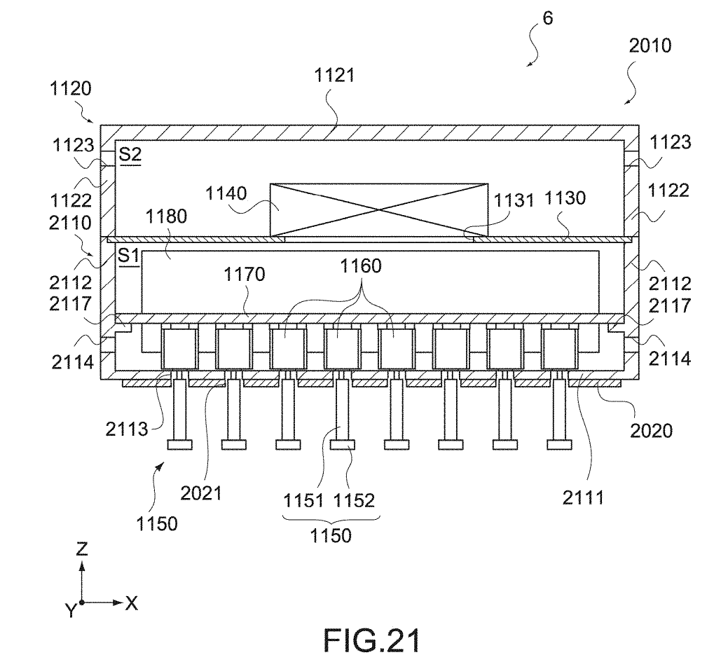

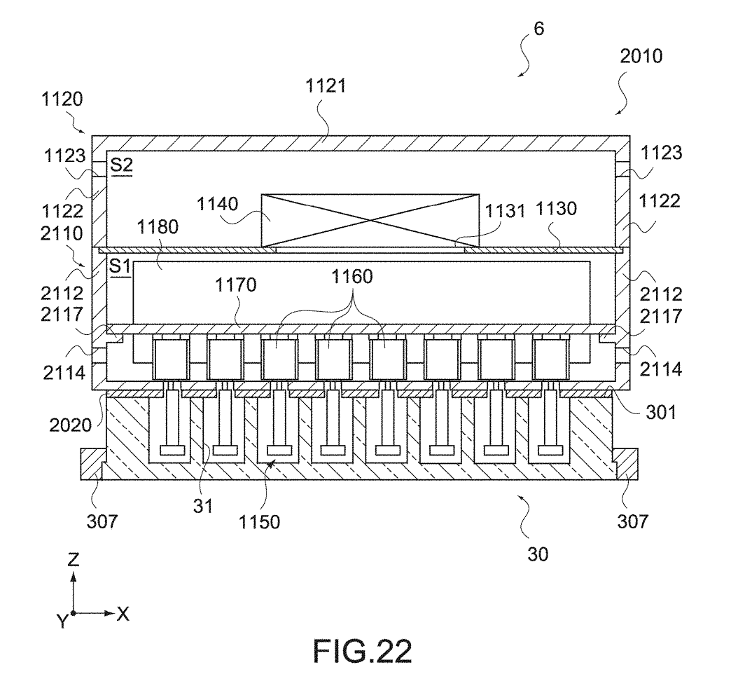

FIG. 20 is an exploded perspective view of a mixing device 6 and a multi-well plate 30 according to a sixth embodiment of the present invention. FIG. 21 is a cross-sectional view of the mixing device 6 along the X-axis direction. FIG. 22 is a cross-sectional view of the mixing device 6 along the X-axis direction in a state of being attached to the multi-well plate 30. Hereinafter, a configuration different from the fifth embodiment will be mainly described, and configurations similar to the embodiments described above will be denoted by similar reference symbols and description thereof will be omitted or simplified.

It should be noted that in each figure the X- and Y-axis directions represent horizontal directions orthogonal to each other, and the Z-axis direction represents a height direction orthogonal to those directions.

[Overall Configuration]

As shown in FIGS. 20 and 21, the mixing device 5 includes a mixing unit 2010 and a sheet member 2020. Further, the mixing device 6 includes a controller that is not shown in the figures as in the first embodiment.

The mixing unit 2010 is configured to be attachable to the multi-well plate 30. The mixing unit 2010 includes a plurality of stirrers 1150 for mixing a solution housed in wells 31 of the multi-well plate 30.

In this embodiment, the mixing unit 2010 includes the plurality of stirrers 1150 corresponding to the wells of the multi-well plate 30, but the mixing unit 2010 is not limited thereto. The mixing unit 2010 may include at least one stirrer.

The sheet member 2020 is a sheet-like member made of an elastic material, which is disposed between the mixing unit 2010 and the multi-well plate 30 and includes through-holes 2021. The through-holes 2021 are holes that penetrate the front and rear surfaces of the sheet member 2020 as shown in FIG. 21, and are provided to the respective wells 31 of the multi-well plate 30 one by one.

A constituent material of the sheet member 2020 is not particularly limited if the constituent material is a material that has heat resistance and chemical resistance and can elastically come into contact with an upper surface 301 of the multi-well plate 30 and the mixing unit 2010, and is typically a silicone rubber.

As in the first embodiment, the controller is for controlling drive of the mixing unit 2010. The controller is electrically connected to the mixing unit 2010 and is configured so as to individually or commonly control rotations of motors that drive the stirrers 1150.

In this embodiment, as shown in FIG. 20, a positioning base 306 is mounted to the multi-well plate 30. The positioning base 306 is detachable from the multi-well plate 30. The positioning base 306 abuts on an outer circumferential surface of the multi-well plate 30 and is fixed to the multi-well plate 30.

The positioning base 306 is provided with positioning holes 307. The number of positioning holes 307 or the shape thereof is not particularly limited, but four positioning holes 307 can be provided on the circumference of the positioning base 306. A constituent material of the positioning base 306 is not particularly limited and can be made of a synthetic resin, for example.

[Mixing Unit]

Details of the mixing unit 2010 will be described. The mixing unit 2010 includes a first casing 2110, a second casing 1120, a fan mounting plate 1130, a fan 1140, stirrers 1150, motors 1160, motor retaining plates 1170, and circuit substrate 1180. The configurations other than the first casing 2110 are similar to those in the fifth embodiment and description thereof will thus be omitted.

The first casing 2110 is made of, for example, a metal material such as an aluminum alloy. The first casing 2110 includes a main surface portion 2111 that faces the upper surface 301 of the multi-well plate 30 and a side-wall portion 2112 hanging from the main surface portion 2111.

The main surface portion 2111 includes a plurality of through-holes 2113 as shown in FIG. 21. The through-holes 2113 penetrate the main surface portion 2111. Each stirrer 1150 is inserted into each through-hole 2113.

Further, the first casing 2110 includes vents 2114. The vents 2114 penetrate the side-wall portion 2112 and cause the inside and the outside of the first casing 2110 to communicate with each other. The shape of the vent 1114 or the number thereof is not particularly limited.

As shown in FIG. 20, the side-wall portion 2112 is provided with a positioning-pin support portion 2115. The positioning-pin support portion 2115 is formed to protrude from the side-wall portion 2112 in the Y direction. It should be noted that the positioning-pin support portion 2215 may be formed to protrude from the side-wall portion 2112 in the X direction. The positioning-pin support portion 2115 is provided with positioning pins 2116.

The positioning pins 2116 protrude from the positioning-pin support portion 2115 toward the multi-well plate 30 and are inserted into the positioning holes 307 of the positioning base 306 as shown in FIG. 20. With this configuration, the mixing unit 2010 is positioned with respect to the positioning base 306 and positioned with respect to the multi-well plate 30 via the positioning base 306.

Further, the side-wall portion 2112 includes a motor-retaining-plate support portion 2117. The motor-retaining-plate support portion 2117 protrudes from the side-wall portion 2112 in a housing space and is configured so as to be capable of placing the motor retaining plates 1170 thereon.

[Operation of Mixing Device]

Next, a typical operation of the mixing device 6 configured as described above will be described.

As shown in FIG. 20, the mixing unit 2010 is placed on the upper surface 301 of the multi-well plate 30 and the positioning pins 2116 are inserted into the positioning holes 307. With this configuration, the mixing unit 2010 is positioned with respect to the multi-well plate 30, and the stirrers 1150 are disposed inside the respective wells 31 of the multi-well plate 30.

At that time, as shown in FIG. 22, the sheet member 2020 comes into close contact with the upper surface 301 of the multi-well plate 30 and the main surface portion 2111 of the first casing 2110.

With this configuration, gaps between the adjacent wells 31 are shielded by the sheet member 2020, and airborne droplets generated by mixing are prevented from being admixed in other wells 31. Further, the sheet member 2020 improves airtightness of each of the wells 31. This suppresses evaporation of the solution in the wells 31.

In this embodiment, the mixing unit 2010 is positioned with respect to the multi-well plate 30 by the positioning pins 2116 of the mixing unit 2010. With this configuration, each of the stirrers 1150 is also disposed in each of the wells 31 with high position accuracy. With this configuration, since the plurality of stirrers 1150 can be collectively positioned with respect to the plurality of minute wells, mixing treatment of the solution in each well can be made uniform.

Furthermore, as in the fifth embodiment, in this embodiment, the fan 1140 is driven by the controller, and an airflow flowing in the internal space S1 from the internal space S2 via the opening 1131 is generated. Further, the first casing 2110 and the second casing 1120 are respectively provided with the vents 2114 and the vents 1123. Therefore, air flows in the internal space S2 from the outside of the mixing unit 1010 via the vents 1123, flows in the internal space S1 by the fan 1140, and is then discharged from the internal space S1 to the outside of the mixing unit 2010 via the vents 2114.

With this configuration, an airflow that flows in from the outside of the mixing unit 2010 and flows out again to the outside of the mixing unit 2010 is generated around the motors 1160 housed in the internal space S1, and the motors 1160 are cooled by the airflow.

It should be noted that the orientation of the discharge of the fan 1140 may be opposite to the above. In this case, air flows in the internal space S1 from the outside of the mixing unit 1010 via the vents 2114, flows in the internal space S2 by the fan 1140, and is then discharged from the internal space S2 to the outside of the mixing unit 2010 via the vents 2114.

In such a manner, the heat generated when the motors 1160 are driven is cooled also by the airflow generated by the fan 1140, in addition to heat transfer to the first casing 2110 and the second casing 1120 that are made of metal. With this configuration, the heat transfer to the multi-well plate 30 can be suppressed, and evaporation, transformation due to the heat, or the like of the solution in the wells 31 can be suppressed.

It should be noted that the vents 1123 and the vents 2114 are not necessarily provided. For example, another opening may be provided at a position different from the opening 1131 of the fan mounting plate 1130. In this case, air flowing from the internal space S2 to the internal space S1 via the opening 1131 by the fan 1140 goes back to the internal space S2 via the other opening. In other words, an airflow circulating inside the mixing unit 1010 is generated, and the motors 1160 are cooled. It should be noted that this embodiment can also have a motor surrounding structure similar to that of the fifth embodiment.

### DESCRIPTION OF REFERENCE NUMERALS

1, 2, 3, 4, 5, 6 mixing device
10, 40, 60, 70, 1010, 2010 mixing unit
11, 1150 stirrer
12, 1160 motor
15, 1030, 1040, 2020 sheet member
16, 610, 710, 1020, 1115, 2116 mounting portion
20 controller
30 multi-well plate
31 well
50 frame body
100, 400, 600, 700, 1110, 1120, 2110 casing
101, 401, 601, 701, 1111, 2111 main surface portion
612 engaging protrusion

1. A mixing device configured to be attachable to a multi-well plate, the mixing device comprising:
a casing that includes a main surface portion facing an upper surface of the multi-well plate;
at least one stirrer that protrudes from the main surface portion toward a well of the multi-well plate;
a drive portion that is disposed on the casing and rotates the stirrer about an axis thereof; and
a mounting portion that is provided to the casing and is mounted to the multi-well plate to position the casing on the multi-well plate.

2. The mixing device according to claim 1, wherein
the stirrer includes a plurality of stirrers, and
the mounting portion is mounted to the multi-well plate to position the plurality of stirrers into predetermined wells of the multi-well plate.

3. The mixing device according to claim 2, wherein
the stirrer includes a plurality of stirrers disposed to correspond to all wells of the multi-well plate.

4. The mixing device according to claim 2, wherein
the stirrer includes a plurality of stirrers disposed to correspond to a plurality of wells belonging to a predetermined row of the multi-well plate.

5. The mixing device according to claim 1, wherein
the mounting portion includes
a space portion that is configured to be capable of housing the multi-well plate, and
an engaging surface that comes into contact with an outer circumferential surface of the multi-well plate housed in the space portion or a part of the outer circumferential surface.

6. The mixing device according to claim 1, wherein
the mounting portion includes a plurality of engaging protrusions that are configured to be engaged with predetermined wells of the multi-well plate.

7. The mixing device according to claim 1, further comprising
a sheet member that is provided to the main surface portion and can elastically come into contact with the upper surface of the multi-well plate.

8. The mixing device according to claim 2, wherein
the drive portion includes a plurality of motors that are respectively attached to the plurality of stirrers.

9. The mixing device according to claim 8, further comprising
a controller that is configured to individually control drive of the plurality of motors.

10. The mixing device according to claim 1, wherein
the mounting portion is a frame body configured to be separable from the casing, and
the frame body has an inner circumferential surface that is engageable with an outer circumferential portion of the casing and an outer circumferential portion of the multi-well plate.

11. The mixing device according to claim 1, wherein
the drive portion is disposed in an internal space of the casing, and
the mixing device further comprises a fan that is disposed in the internal space.

12. The mixing device according to claim 11, further comprising
a fan mounting plate that partitions the internal space into a first internal space and a second internal space and has an opening that causes the first internal space and the second internal space to communicate with each other, the first internal space housing the drive portion, the second internal space housing the fan, the fan generating an airflow flowing between the first internal space and the second internal space via the opening.

13. The mixing device according to claim 12, wherein
the casing includes a first vent hole and a second vent hole, the first vent hole causing the first internal space and an outer space of the casing to communicate with each other, the second vent hole causing the second internal space and the outer space to communicate with each other.

14. The mixing device according to claim 1, wherein
the mounting portion is an attachment configured to be separable from the casing.

15. The mixing device according to claim 1, wherein
the mounting portion is mounted to the multi-well plate via a positioning member mounted to the multi-well plate.

16. The mixing device according to claim 1, further comprising
a sheet member that elastically comes into contact with the main surface portion, wherein the drive portion includes a chassis, a rotary shaft, and a bearing, the chassis housing a rotor and a stator, the rotary shaft being connected to the rotor, the bearing being fixed to the chassis and rotatably supporting the rotary shaft,
the stirrer is connected to the rotary shaft, and
the mixing device further comprises a sealing that seals a gap between the sheet member and the stirrer.

17. The mixing device according to claim 1, further comprising
a sheet member that elastically comes into contact with the main surface portion, wherein the drive portion includes a chassis, a rotary shaft, and a bearing, the chassis housing a rotor and a stator, the rotary shaft being connected to the rotor, the bearing being fixed to the chassis and rotatably supporting the rotary shaft,
the stirrer is connected to the rotary shaft, and
the casing seals a gap between the sheet member and the stirrer.

18. The mixing device according to claim 1, further comprising
a controller that controls the drive portion, the controller controlling the drive portion to generate a first torque for a certain period of time when the drive portion starts to rotate and controlling the drive portion to alternately generate a second torque and the first torque after the certain period of time elapses, the second torque being smaller than the first torque.
